(19) United States
(12) Patent Application Publication
(10) Pub. No.: US 2022/0023213 A1
(43) Pub. Date: Jan. 27, 2022

(54) NANOCRYSTALLINE EYE DROP, PREPARATION METHOD AND USE THEREOF

(71) Applicant: CHENGDU RUIMU PHARMACEUTICALS CO., LTD., Chengdu, Sichuan (CN)

(72) Inventor: Qing DONG, Chengdu, Sichuan (CN)

(21) Appl. No.: 17/294,050

(22) PCT Filed: Dec. 24, 2019
(86) PCT No.: PCT/CN2019/127735

§ 371 Date: May 14, 2021

(30) Foreign Application Priority Data
Nov. 19, 2018 (CN) 201811379085.3
Nov. 18, 2019 (CN) 201911129988.0

(51) Int. Cl. A61K 9/127; A61K 47/20; A61K 47/34; A61K 47/38; A61K 45/06; A61K 9/00

(52) U.S. Cl. CPC A61K 9/1274; A61K 47/20; A61K 47/34; B82Y 5/00; A61K 45/06; A61K 9/0048; A61K 47/38

(57) ABSTRACT

A nanocrystalline eye drop contains a double-soluble macromolecule, a single-soluble macromolecule, and a fat-soluble drug. The double-soluble macromolecule and the single-soluble macromolecule interact with each other to encapsulate the fat-soluble drug to form and stabilize a nanocrystalline. The drug can rapidly pass through a blood-ocular barrier into a vitreous body by means of special intercellular space infiltration and/or pinocytosis, and achieve an effective therapeutic effect by means of passive targeting and attachment.

### TECHNICAL FIELD

The present invention relates to the field of eye drops, and in particular to a nanocrystalline eye drop, a preparation method and a use thereof.

### BACKGROUND

When an eye drop is put onto an eye for the treatment of a disease, most of the drug is instilled in the lower conjunctival fornix. Then a motion of blinking allows the drug to spread through capillaries into the anterior membrane of the cornea and thus penetrate into the cornea. The drug must penetrate through the blood-ocular barriers smoothly to enable the ocular tissues to achieve a proper drug concentration. But corneal epithelial cells, the blood-aqueous barrier and the blood-retina barrier prevent the penetration of the drug, leading to a reduction in the absorption and therapeutic effect of the drug.

In the prior art, the method of intravitreal injection is often adopted to get the drug into the fundus and maintain an effective concentration there. For example, for the treatment of macular degeneration, the vascular endothelial growth factor (VEGF) receptor antagonists including Ranibizumab, Bevacizumab, Aflibercept and Conbercept need to be administered by intravitreal injection for a long term. However, eye injections are risky. Intravitreal injection poses a potential risk of ocular tissue damage, retinal detachment, hemorrhage, intraocular pressure elevation and endophthalmitis. In addition, these drugs have common side effects including blood pressure elevation, vascular death and cerebrovascular accidents (HATA M. et al., RETINA 2017, 37:1320). To reduce the frequency of injections, a high dose of such a drug is often injected at a time. But an excessive dosage may interfere with the normal VEGF levels in the blood. Therefore, the total exposure to anti-VEGF drugs needs to be considered carefully, especially when treating with high-risk patients whom have vascular diseases. According to a meta-analysis of four clinical studies, the monthly injections of Aflibercept or 0.5 mg Ranibizumab into patients with DME (diabetic macular Edema) for two years yield an increased risk for death, vascular death or cerebrovascular accidents compared with sham and laser treatments (JAMA Ophthalmol. 2016; 134(1): 21).

Furthermore, the administration of drugs by intravitreal injection must be carried out by well-trained medical staff in a qualified hospital. The treatment and examination methods are complex with high costs, and put heavy economic burdens on patients. Besides, poor patient compliance also affects the outcomes of long-term therapy. As most patients with these eye diseases are seniors, frequent intravitreal injections can not only increase their economic burdens but also increase the risk of adverse reactions including endophthalmitis. This method is inconvenient for senior patients and poses potential risks.

Many new drug research and development organizations around the world have been trying to develop new drugs which do not require intravitreal injections for the treatment of fundus oculi diseases. In the US, clinical studies were carried out on the treatment of wet AMD (age-related macular degeneration) with squalamine eye drop (OHR-102). The early clinical data of these studies demonstrated were efficacy, but phase III studies did not achieve the desired purposes (Retinal Physician, Issue: January/February 2018). In the US and China, clinical studies were conducted on the treatment of wet AMD with Vorolanib (X-82, CM082), an oral tyrosine kinase inhibitor. It was observed in the early dose-escalation clinical tests (an oral dose of 50-300 mg/day) that X-82 could maintain or improve the eyesight of participants (Jackson et al, JAMA Ophthalmol. 2017; 135:761), but the long-term administration of the anti-cancer drugs may increase the risk of systemic toxic and side effects in patients. Ruan, Tan, et al made eye drops of tinibs only for the treatment of ocular surface angiogenesis-related diseases such as pterygium. Such an eye drop could inhibit the increase of ocular surface angiogenesis in a rabbit eye suture model. But they did not study the fundus angiogenesis-related diseases.

Therefore, to provide an eye drop, which needs no vitreous injection, be able to penetrate into vitreous body and reach an effective drug concentration in the fundus has been a big urgent challenge need to be solved by those skilled in the art.

### SUMMARY

A first purpose of the present invention is to provide a nanocrystalline eye drop, which can penetrate through a blood-ocular barrier into a vitreous body, reach an effective concentration at a fundus, and be convenient to use.

A second purpose of the present invention is to provide a preparation method for the nanocrystalline eye drop, which is simple to operate with mild reaction conditions and allows quick preparation of the nanocrystalline eye drop.

A third purpose of the present invention is to provide a use of the nanocrystalline eye drop, which widens applications of the eye drop in the treatment of the diseases of fundus oculi and solves the current clinical problem of fundus oculi disease treatment requiring intravitreal injection.

An eyeball is a special structure composed of anterior and posterior segments. The anterior segment, i.e. an ocular surface, contains tears. The eyeball surface is covered with a tear film, and anterior is composed of a lipid layer, an aqueous layer and a mucin layer. Both epithelium and endothelium of a cornea contain abundant lipids. A drug needs to penetrate through the aqueous stromal layer of the cornea first, then through the oily lipid layer before reaching the fundus; however, it is hard for a dissociable drug to penetrate through an intact cornea. It is a huge challenge to enable the active pharmaceutical ingredient (API) to pass through a series layers of two-type of liquid phases with opposite polarities i.e. the aqueous phase and the oil phase, to reach the vitreous body. No report has been revealed as a drug delivery eye drop be able to administrate to the posterior segment and treat fundus angiogenesis-related diseases.

In the present invention, a special new nanocrystalline eye drop is designed and developed creatively in light of the particular structures of the eyes and their barriers that drugs need to overcome to reach the fundus after the instillation. This new eye drop can penetrate through the blood-ocular barriers into the vitreous body and achieve therapeutic concentration in the fundus.

A fat-soluble drug is used as API in the present invention. More than one excipient, i.e. double-soluble macromolecule and a single-soluble macromolecule, are selected in order to achieving compatibility with the API, so that a nanoparticle that has a hydrophilic exterior and a fat-soluble API-based core can be formed during physical dispersion in a medium. The combination of a double-soluble macromolecule that is soluble in both aqueous and organic phases, and a single-soluble macromolecule as the excipients is enable to making a fat-soluble API into aqueous solution of nanocrystalline. After instilled into the eyes, this solution will not be rejected by tears. When nanocrystalline particles touch the ocular surface, the API is able to attach to the lipid layer of the ocular surface and thus reaches the fundus by means of infiltration and/or pinocytosis.

The technical solution of the present invention is as follows:

The present invention relates to a nanocrystalline eye drop comprising a double-soluble macromolecule, a single-soluble macromolecule and a fat-soluble drug;

the double-soluble macromolecule and the single-soluble macromolecule interact with each other to encapsulate the fat-soluble drug to form nanocrystallines and maintain their stabilities.

Due to the hydrophilic property of a double-soluble macromolecule, the eye drop in the present invention is affiliative to the aqueous phase on the ocular surface. As the fat-soluble drug is affiliative to the lipid phase after touching the ocular surface, it is helpful for the eye drop to permeate the focus on the fundus or the vitreous body. The rejection of the ocular aqueous layer to water-insoluble (fat-soluble) drugs is overcome by the synergistic effects of the double-soluble macromolecule and the single-soluble macromolecule. Once this eye drop touching the ocular surface, the nanocrystalline particles of the drug may rupture, and the fat-soluble drug will enter into the lipid layer on the ocular surface with the aid of macromolecular substances and then gradually reach the posterior eye segment.

The nanocrystalline particles of the micromolecular drug are small by size, which is also good for the penetration into the posterior segment.

In the technical solution of the present invention, the eye drop is a solution or a suspension.

In the technical solution of the present invention, the described fat-soluble drug comprises a targeting drug acting on a vascular endothelial growth factor receptor (VEGFR) and/or a platelet-derived growth factor receptor (PDGFR).

The fat-soluble drug used in the present invention comprises a targeting drug acting on VEGFRs and/or PDGFRs.

Furthermore, the targeting drugs are tyrosine kinase inhibitors. Preferably, the tyrosine kinase inhibitors are selected from any one or more of the tinibs and their medical acceptable salts, or more preferably, from any one or more of axitinib, semaxanib, sorafenib, regorafenib, pazopanib, vandetanib and sunitinib.

In the present invention, the creative use of tinibs as the API of an eye drop enables the drug to reach the fundus under the effects of a double-soluble macromolecule and a single-soluble macromolecule and to act on the fundus blood vessels for treatment. The present invention provides a solution to the problem that no VEGFR- and PDGFR-targeting drugs have been used for the treatment of ophthalmic diseases and also extending tinibs medical applications. In addition, tinibs are small molecular drugs, with higher tissue permeability comparing to bio-macromolecular drugs, thus making them easier to enter the fundus.

It should be noted that the example of the present invention only exemplifies a part of the tinibs, and the rest of them, as well as their medicinal acceptable salts can also be used as APIs.

In the technical solution of the present invention, the double-soluble macromolecule is a macromolecule stabilizer containing both hydrophilic and fat-soluble groups, so that the double-soluble macromolecule has a good affinity to the aqueous phase at ocular surface, as well be able properly encapsulate the fat-soluble drug.

Specifically, the double-soluble macromolecule is a surfactant. More preferably, it can be any one or at least two of the following, poloxamers, tweens, sodium dodecyl compounds, polyvinylpyrrolidones and polyethylene glycol compounds.

Preferably, the sodium dodecyl compound is sodium dodecyl sulfonate and/or lauryl sodium sulfate; The polyethylene glycol compound is any one or more of PEG4000, PEG5000 or PEG6000.

In the present invention, the double-soluble macromolecule interacts with the single-soluble macromolecule to perform encapsulation, while serves as a stabilizer, to prevent the nanocrystalline eye drop from precipitating or growing, thereby ensuring the therapeutic effect of the drug.

In the technical solution of the present invention, the single-soluble macromolecule is a macromolecular suspending agent or a co-solvent comprising hydrophilic or fat-soluble groups, thus making the single-soluble macromolecule soluble in either water or fat-soluble solvent.

Specifically, the single-soluble macromolecule is any one or at least two of the following, starches, celluloses and polycarboxylate compounds;

More preferably, the cellulose is any one or at least two of the following, chitosan, hyaluronic acid (HA), methyl cellulose, carboxyl methyl cellulose (CMC), hydroxypropylcellulose (HPC), hydroxypropyl methyl cellulose (HPMC), sodium carboxyl methyl cellulose (CMC-Na).

The starches comprise any one or at least two of the following, sodium carboxyl methyl starch, amylose and dextrin;

The polycarboxylate compounds are any one or at least two of the following, polylactic acid (PLA), polyglycolic acid (PGA), and poly(lactic-co-glycolic acid) (PLGA).

In the technical solution of the present invention, the single-soluble macromolecule serves not only as a part that encapsulates the drug, but also a suspending role, thereby improving the stability and ensuring the therapeutic effects of the drugs.

It should be noted that the hydrophilic substitute groups described in the present invention include, but not limited to carboxylic acid group, sulfonic acid group, phosphoric acid group, amino group, quaternary ammonium group, ether bond, hydroxyl group, and carboxylic ester. Fat-soluble group includes, but not limited to, aliphatic group, aromatic group, higher fatty hydroxyl group and carbalkoxy.

The interaction between the single-soluble macromolecule and the double-soluble macromolecule not only wraps the drug, but also improves the stability of the drug and prevents the drug from precipitating or growing.

In the technical solution of the present invention, the mass ratio of the double-soluble macromolecule to the single-soluble macromolecule is 1-5:1, preferably 1-2:1.

The above ratio ensures that the interaction between the single-soluble macromolecule and the double-soluble macromolecule properly wraps the drug, while prevents the nanocrystalline from precipitating or growing and enables the nanocrystalline to be absorbed effectively.

In the technical solution of the present invention, the mass ratio of the double-soluble macromolecule to the fat-soluble drug is 2-12:1, preferably 5-10:1.

In the technical solution of the present invention, the content of the fat-soluble drug in the nanocrystalline eye drop is 0.06-100 mg/mL.

Based on quite a number of experiments and creative efforts made by the inventor, the mass ratio of the double-soluble macromolecule to a fat-soluble drug and the content of the fat-soluble drug is determined. The ratio ensures a proper drug concentration in the nanocrystalline eye drop, the therapeutic effect of the nanocrystalline eye drop, the encapsulation of the fat-soluble drug by the double-soluble macromolecule and the single-soluble macromolecule, as well as the absorptivity of the drug.

In the technical solution of the present invention, the particle size of the nanocrystalline in the nanocrystalline eye drop is 200-1000 nm, preferably 300-800 nm. To ensure the stability of the nanocrystalline in the nanocrystalline eye drop, it is necessary to control the particle size of nanocrystalline. If the particle size is too big, the drug efficacy can be compromised due to lack of the unique permeability of nano-drugs. On the contrary, if the particle size is too small, the drug is prone to aggregate and precipitate. Through a great deal of experiments, the inventor accidentally found that the drug is desirably permeable and not easy to aggregate and precipitate if the particle size is within the above range.

It should be noted that the particle size in the present invention refers to the average particle size or the particle size of most nanocrystallines. In the nanocrystalline eye drop, there may also be submicron crystals with particle size between 1 and 3 μm.

The present invention also provides a method for preparing the nanocrystalline eye drop, comprising the following steps: after mixing the double-soluble macromolecule, the single-soluble macromolecule and the fat-soluble drug, reducing the particle size of the drug to form the stable, encapsulated nanocrystalline.

Specifically, mixing the double-soluble macromolecule and the single-soluble macromolecule to form a mixed solution; and then mixing the mixed solution and the fat-soluble drug to form an initial suspension; grinding or homogenizing the initial suspension to form the nanocrystalline eye drop that stably encapsulates the fat-soluble drug.

In the example of the present invention, the double-soluble macromolecule and the single-soluble macromolecule are mixed with water to form the mixed solution and then the mixed solution is mixed with the fat-soluble drug to form the initial suspension.

In the mixed solution, the content of double-soluble macromolecule per 100 mL of water is 4-1000 mg; and/or the content of single-soluble macromolecule per 100 mL of water is 4-1000 mg;

preferably, the content of the double-soluble macromolecule per 100 mL of water is 10-300 mg.

The present invention can lower the particle size of materials in the initial suspension through the grinding or homogenizing operation, and to ensuring the encapsulation effect, which allow the drug to pass through the blood-ocular barrier and enter the vitreous body easier by means of intercellular space infiltration and/or pinocytosis, thus improving the utilization ratio and therapeutic effect of the drug.

The grinding in the present invention is carried out under a low temperature. Specifically, the drug is ground for 1-3 h at a speed of 300-500 rpm under the temperature of 0° C.-5° C. The grinding container is a sealed cup made of zirconia. The grinding beads are also made of zirconia, with a particle size of 0.1-0.2 mm or 0.3-0.4 mm.

It should be noted that the particle size of the drug can also be reduced by other modes in the prior art such as high-pressure homogenization and mechanical shearing, in addition to the method provided in the examples of the present invention.

The present invention also provides the application of a nanocrystalline eye drop in the preparation of a drug used for treating fundus oculi diseases or/and ocular surface diseases.

Specifically, the fundus oculi diseases comprise diseases related to fundus neovascularization, and the ocular surface diseases comprise diseases related to ocular surface neovascularization;

Preferably, the diseases related to fundus neovascularization comprise any one or several ones of age-related macular degeneration, retinal vein occlusion macular edema, central retinal vein occlusion, diabetic retinopathy, diabetic macular edema, or the impaired vision, neovascular glaucoma and eye tumors caused by choroidal neovascularization secondary to pathological myopia;

Preferably, the diseases related to ocular surface neovascularization comprise any one or several ones of viral keratitis, corneal neovascularization caused by physical and/or chemical trauma, corneal transplantation, corneal neovascularization, ocular surface neovascularization and pterygium, corneal neovascularization complicated by pterygium, corneal neovascularization due to corneal transplantation rejection, and deficiency of corneal stem cells.

The experiment of animal drug absorption vitreous body shows that the effective dose of the drug of the present invention can reach the fundus vitreous body through the blood-ocular barrier, and in most cases the concentration in the vitreous body of animals reaches the maximum absorption at 30-60 minutes after instillation.

In the animal pharmacological experiment, a classical laser-induced coagulation neovascularization (CNV) model animal eye is adopted. The experiment shows that the drug provided in the present invention can effectively inhibit the fundus neovascularization.

Compared with the prior art, the present invention has the following beneficial effects:

The present invention creatively designs a nanocrystalline eye drop, which can reach the effective drug concentration at fundus by passing the blood-ocular barrier and entering the vitreous body, featuring safety and convenience of drug use. It allows the drug to enter the vitreous body to treat fundus oculi disease while the drug takes effect on the ocular surface to treat ocular surface diseases, avoiding the high risk of intravitreal injection, improving the therapeutic compliance and effect, and reducing the treatment cost.

In the present invention, it is available to encapsulate the drug through interaction between the double-soluble macromolecule and the single-soluble macromolecule, so as to prevent drug aggregation and ensure the drug stability. Furthermore, the drug can pass through a blood-ocular barrier into the vitreous body by means of special intercellular space infiltration and/or pinocytosis, thus enhancing the utilization ratio of drug and improving the therapeutic effect by means of passive targeting and attachment.

Due to the affinity to the aqueous phase on the ocular surface and the lipid phase after contact with the ocular surface, the nanocrystalline eye drop in the present invention is easier to infiltrate the focus on fundus vitreous body. In addition, the nanocrystalline particles with a suitable particle size are not only conducive to the drug stability, but also help the drug infiltrate and reach the posterior eye segment.

The macromolecular excipients selected in the present invention feature good biocompatibility, enabling the solution of API, the enhancing permeability of the drug particles in eye tissues, and good for API getting into the posterior eye segment.

In the present invention, the creative use of tinibs as the API of an eye drop provides a solution to the problem that no VEGFR- and PDGFR-targeting drugs have been used in eye drops, and also widens its range of application. In addition, tinibs are small molecular drugs, with tissue permeability higher than that of bio-macromolecular drugs, thus making it easier to enter the fundus.

The pharmacodynamic test shows that the nanocrystalline eye drop according to the present invention can pass through the blood-ocular barrier to the fundus vitreous body to play a therapeutic role.

### BRIEF DESCRIPTION OF THE DRAWINGS

In order to more clearly illustrate the technical solution in the examples of the present invention or in the prior art, a brief introduction of the accompanying drawings that are required to describe the example is given below.

FIG. 1 shows an SEM phenogram of a drug of a nanocrystalline eye drop in Example 1.

FIG. 2 shows an SEM phenogram of a drug of a nanocrystalline eye drop in Example 2.

FIG. 3 shows an SEM phenogram of a drug of a nanocrystalline eye drop in Example 3.

FIG. 4 shows an SEM phenogram of a drug of a nanocrystalline eye drop in Example 4.

FIG. 5 shows a fluorescence contrast image of eyes of animals in a pharmacodynamic test in which a CNV model is built for laser-induced mice eyes.

### DETAILED DESCRIPTION OF THE PREFERRED EXAMPLES

To make the objectives, technical solutions, and advantages of the examples of the present invention clearer, the technical solution in the examples of the present invention will be described clearly and completely below. Unless otherwise specified in the examples, the examples were carried out according to conventional conditions or the conditions recommended by manufacturers. Reagents or instruments used, without specific manufacturers, are conventional products purchased from the market.

Further detailed descriptions to the characteristics and performances of the present invention are made in combination with the examples.

### Example 1

This example discloses a nanocrystalline eye drop and a preparation method thereof according to the present invention.

The nanocrystalline eye drop of this example includes a double-soluble macromolecule, a single-soluble macromolecule and a fat-soluble drug. The double-soluble macromolecule and the single-soluble macromolecule interact with each other to encapsulate the fat-soluble drug.

The double-soluble macromolecule is poloxamer 188; the single-soluble macromolecule is HPC μF; and the fat-soluble drug is axitinib as a targeting drug, with the effects on both a vascular endothelial growth factor receptor and a platelet-derived growth factor receptor.

The mass ratio of poloxamer 188 to HPC μF is 5:1, and that of poloxamer 188 to axitinib is 10:1.

The preparation method of the nanocrystalline eye drop according to this example includes the following steps of:

dispersing 0.5 g poloxamer 188 and 0.1 g HPC μF into 50 mL of purified water, and preparing a mixed solution through heating and stirring;
dispersing 50 mg axitinib into the mixed solution to prepare an initial suspension with a concentration of 1 mg/mL;
transferring the initial suspension into a planetary ball mill for rapid grinding for 2 h at 350 rpm under temperature of 0° C. A grinding container is a 100 mL sealing cup made of zirconia; a grinding bead is a spherical bead made of zirconia, with a particle size of approximately 0.3-0.4 mm. A finished product prepared through decompression filtration via a filter membrane is an axitinib drug nanosuspension.

### Examples 2-9

A nanocrystalline eye drop and a preparation method thereof according to the present invention are provided in the Examples 2-9.

When the nanocrystalline eye drops provided in the Examples 2-9 are compared with that provided in the Example 1, a double-soluble macromolecule, a single-soluble macromolecule and a fat-soluble drug have the same type. The prepared nanocrystalline eye drops have the same structure, with a difference in specific compounds used, or/and the mass ratio of the compounds.

The preparation methods of the nanocrystalline eye drops provided in the Examples 2-9 are basically the same as that in the Example 1, with a difference in operating conditions.

### Example 2

This example discloses a nanocrystalline eye drop and a preparation method thereof according to the present invention.

In the nanocrystalline eye drop, the double-soluble macromolecule is poloxamer 188; the single-soluble macromolecule is HPC μF; the fat-soluble drug is axitinib; the mass ratio of the double-soluble macromolecule to the single-soluble macromolecule is 5:1; and that of poloxamer 188 to axitinib is 5:1.

A process nanocrystalline eye drop is prepared under conditions of grinding temperature of 0° C., a rotation speed of 350 rpm and grinding time of 2 h.

### Example 3

This example discloses a nanocrystalline eye drop and a preparation method thereof according to the present invention.

In the nanocrystalline eye drop, the double-soluble macromolecule is Tween 80; the single-soluble macromolecule is HPC μF; the targeting drug is axitinib; the mass ratio of the double-soluble macromolecule to the single-soluble macromolecule is 5:1; and that of Tween 80 to axitinib is 10:1.

A process nanocrystalline eye drop is prepared under conditions of grinding temperature of 3° C., a rotation speed of 350 rpm and grinding time of 1.5 h.

### Example 4

This example discloses a nanocrystalline eye drop and a preparation method thereof according to the present invention.

In the nanocrystalline eye drop, the double-soluble macromolecule is Tween 80; the single-soluble macromolecule is HPMC E5; the targeting drug is axitinib; the mass ratio of the double-soluble macromolecule to the single-soluble macromolecule is 5:1; and that of Tween 80 to axitinib is 10:1. A process nanocrystalline eye drop is prepared under conditions of grinding temperature of 5° C., a rotation speed of 350 rpm and grinding time of 3 h.

### Example 5

This example discloses a nanocrystalline eye drop and a preparation method thereof according to the present invention.

In the nanocrystalline eye drop, the double-soluble macromolecule is a mixture of PEG4000, PEG5000 and sodium dodecyl sulfonate; the single-soluble macromolecule is a sodium carboxymethyl starch; the targeting drug is regorafenib; the mass ratio of the double-soluble macromolecule to the single-soluble macromolecule is 3:1; and that of the double-soluble macromolecule to regorafenib is 12:1.

A process nanocrystalline eye drop is prepared under conditions of grinding temperature of 2° C., a rotation speed of 350 rpm and grinding time of 2.5 h.

### Example 6

This example discloses a nanocrystalline eye drop and a preparation method thereof according to the present invention.

In the nanocrystalline eye drop, the double-soluble macromolecule is poloxamer 188; the single-soluble macromolecule is PLGA; the targeting drug is vandetanib; the mass ratio of the double-soluble macromolecule to the single-soluble macromolecule is 1:1; and that of poloxamer 188 to vandetanib is 5:1.

A process nanocrystalline eye drop is prepared under conditions of grinding temperature of 0° C., a rotation speed of 500 rpm and grinding time of 3 h.

### Example 7

This example discloses a nanocrystalline eye drop and a preparation method thereof according to the present invention.

In the nanocrystalline eye drop, the double-soluble macromolecule is a mixture of PEG6000 and Tween 80; the single-soluble macromolecule is a sodium carboxymethyl starch; the targeting drug is regorafenib; the mass ratio of the double-soluble macromolecule to the single-soluble macromolecule is 3:1; and that of the double-soluble macromolecule to regorafenib is 12:1.

A process nanocrystalline eye drop is prepared under conditions of grinding temperature of 2° C., a rotation speed of 350 rpm and grinding time of 2.5 h.

### Example 8

This example discloses a nanocrystalline eye drop and a preparation method thereof according to the present invention.

In the nanocrystalline eye drop, the double-soluble macromolecule is sodium dodecyl sulfate; the single-soluble macromolecule is chitosan; the targeting drug is sorafenib; the mass ratio of the double-soluble macromolecule to the single-soluble macromolecule is 4:1; and that of sodium dodecyl sulfate to sorafenib is 6:1.

A process nanocrystalline eye drop is prepared under conditions of grinding temperature of 3° C., a rotation speed of 450 rpm and grinding time of 1.5 h.

### Example 9

This example discloses a nanocrystalline eye drop and a preparation method thereof according to the present invention.

In the nanocrystalline eye drop, the double-soluble macromolecule is Tween 80; the single-soluble macromolecule is hyaluronic acid; the targeting drug is sunitinib; the mass ratio of the double-soluble macromolecule to the single-soluble macromolecule is 2.5:1; and that of Tween 80 to sunitinib is 8:1. A process nanocrystalline eye drop is prepared under conditions of grinding temperature of 3° C., a rotation speed of 480 rpm and grinding time of 2 h.

### Characterization

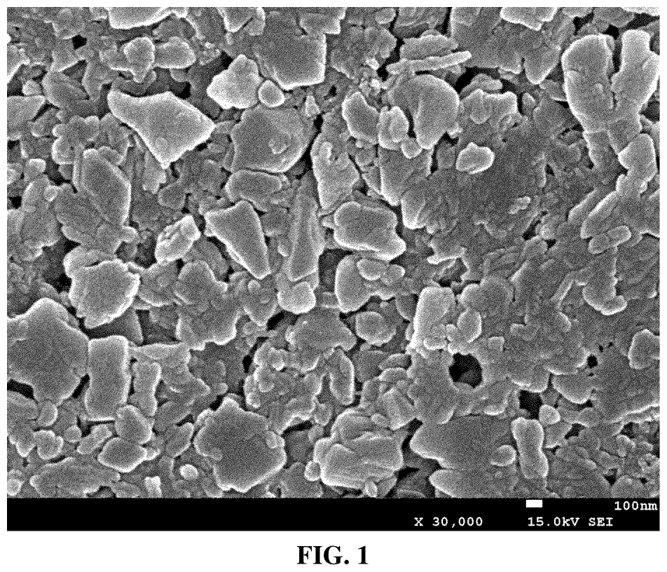

The nanocrystalline eye drops prepared in the Examples 1˜4 are subject to SEM detection, with detection results as shown in FIGS. 1-4.

FIG. 1 is an SEM diagram of the Example 1. Referring to FIG. 1, nanocrystallines in the nanocrystalline eye drop of the Example 1 are flake-like, and a part of the nanocrystallines has an adhesion phenomenon, with a particle size between 100 nm and 800 nm.

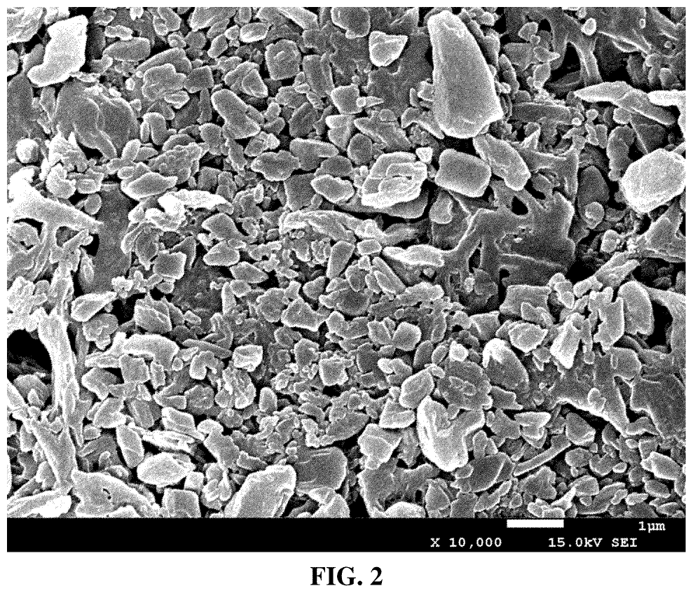

FIG. 2 is an SEM diagram of the Example 2. Referring to FIG. 2, nanocrystallines in the nanocrystalline eye drop of the Example 2 are flake-like, and a part of the nanocrystallines adhesion phenomenon is not obvious, with a particle size between 100 nm and 2 μm and wider distribution in the particle size.

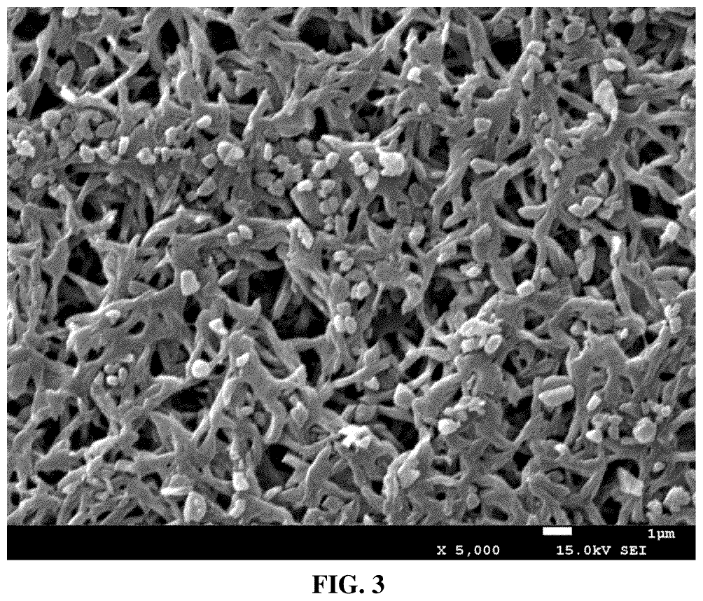

FIG. 3 is an SEM diagram of the Example 3. Referring to FIG. 3, nanocrystallines in the nanocrystalline eye drop of the Example 3 are minor block-shaped particles, with a particle size approximately between 100 nm and 600 nm.

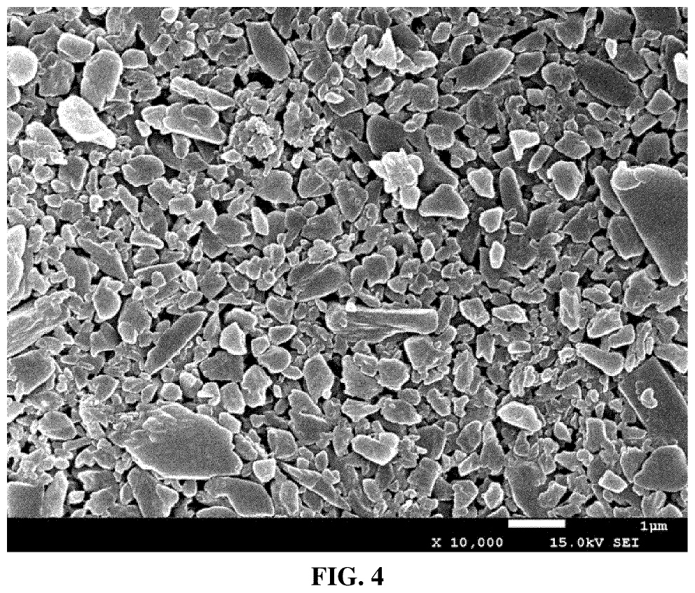

FIG. 4 is an SEM diagram of the Example 4. Referring to FIG. 4, nanocrystallines in the nanocrystalline eye drop of the Example 4 are minor block-shaped particles, with a particle size approximately between 300 nm and 800 nm.

Comparative example 1: the nanocrystalline eye drop prepared according to the preparation method of the Example 1 differs in that the double-soluble macromolecule used is a substance only having a hydrophilic group, namely sodium stearate; and the nanocrystalline eye drop prepared by the double-soluble macromolecule may be a gel status, instead of a nanocrystalline structure.

Comparative example 2: the nanocrystalline eye drop prepared according to the preparation method of the Example 1 differs in that the double-soluble macromolecule used is a substance only having a fat-soluble group, namely glyceryl tristearate; and the nanocrystalline eye drop prepared by the double-soluble macromolecule may be a microspherulitic structure, instead of a nanocrystalline structure.

Comparative example 3: the nanocrystalline eye drop prepared according to the preparation method of the Example 1 differs in that the single-soluble macromolecule used is a substance only having a hydrophilic substrate and an oleophylic substrate, namely lecithin; and the nanocrystalline eye drop prepared by the single-soluble macromolecule may be a gel status, a microspherulitic structure or other structures, instead of a nanocrystalline structure.

Comparative example 4: the nanocrystalline eye drops are prepared according to the preparation method provided in the Example 1, except that the axitinib is ground first, and the grinding conditions are the same as those in the Example 1, and then the ground axitinib is mixed with a mixed solution, but nanocrystallines cannot be obtained by this method.

### Stability Determination

The nanocrystalline eye drops of Example 1 to 4 and comparative examples 1 to 4 are placed at 25±5° C. and a relative humidity of 60±10% for 30 days, and then D90, D50 and D10 of the nanocrystalline eye drops are detected, and detection results are shown in a table 1.

Among them, the apparatus used for detection is Microtrac S3500, the detection conditions are: a wet method, dispersion medium water, a flow rate 60%, ultrasonic power 30 w, and ultrasonic time 120 S; a detection process: setting experiment parameters according to the above experiment conditions; filling a wet sampler with water, starting internal circulation, and starting zero adjustment at the same time; and after the zero adjustment of an instrument is passed, adding prepared nanocrystalline suspension dropwise until the concentration reaches a concentration range specified by the instrument, starting internal ultrasound, and testing PSD results after the ultrasound.

TABLE 1
Detection Results

| | Time | | | | | |
|---|---|---|---|---|---|---|
| | 0 day | | | 30 days | | |
| Particle size distribution | D90(μm) | D50(μm) | D10(μm) | D90(μm) | D50(μm) | D10(μm) |
| Example 1 | 1.312 | 0.522 | 0.244 | 1.51 | 0.544 | 0.245 |
| Example 2 | 1.367 | 0.477 | 0.226 | 1.356 | 0.472 | 0.229 |
| Example 3 | 1.746 | 0.878 | 0.446 | 2.02 | 0.865 | 0.421 |
| Example 4 | 1.887 | 0.932 | 0.437 | 2.815 | 0.974 | 0.412 |

It can be seen from the table 1 that the nanocrystalline eye drops provided by the examples of the present invention have good stability, and drugs are not easy to aggregate. However, since the substances prepared in the comparative examples 1 to 4 are not nanocrystallines, during a storage process, the drugs aggregate quickly and the stability is poor.

The nanocrystalline eye drops of the Example 1 to 4 are placed at 25±5° C. and the relative humidity of 60±10% for 60 days, and then the content of the nanocrystalline eye drops are analyzed. The nanocrystalline eye drops are filtered with a 0.45 μm membrane, and a filtrate is used as a test solution; API is dissolved by adding methanol to prepare a reference solution with API content of 0.1 mg/ml. The content is determined by an external standard method. Specific detection conditions are shown in Table 2, and specific detection results are shown in Table 3.

TABLE 2
Analyses Conditions
Chromatographic conditions

| | Agilent 1100 high performance |
|---|---|
| Apparatus | liquid chromatograph system |
| Chromatographic | Agilent Eclipse XDB-C18 4.6 × 150 mm, 5 μm |
| column | |
| Detection | 260 nm |
| wavelength | |
| Mobile phase | 20 mM phosphate buffer(pH = 3.0) − |
| | acetonitrile (40:60, v/v) |
| Flow rate | 1.0 ml/min |
| Column temperature | 35° C. |
| External standard | 0.1 API mg/ml (methanol) |
| Injection volume | 10 μl |
| Retention time | 1.94 min |

TABLE 3
Detection Results

| | API concentration | | Relative |
|---|---|---|---|
| Sample No. | mg/mL | Peak area | percentage % |
| Example 1 | 0.1 | 4684 | 99.34 |
| Example 2 | | 4627 | 98.14 |
| Example 3 | | 4640 | 98.41 |
| Example 4 | | 4629 | 98.18 |
| Raw material | | 4687 | 99.41 |
| External standard | | 4715 | 100 |
| substance | | | |

It can be seen from the table 3 that the nanocrystalline eye drops provided by the examples of the present invention have good stability, and the effective content of the drugs can be guaranteed.

### In Vivo Animal Experiment

### Example 10

### Pharmacodynamic Experiment

Inhibition experiments to rabbit ocular surface alkali burnt corneal neovascularization (CNV) by using the drugs prepared in the Examples 1 and 4 are applied.

Ten New Zealand male rabbits, 2.0 to 2.5 kg, 3 to 4-month-old are divided into 1 normal control group (one animal and two eyes); 3 experiment groups (a model group, an Example 1 group and an Example 4 group), each group has 3 animals and 6 eyes, their corneas were burnt with 1 mol/L NaOH solution, CNV observed significantly. On the first day (Day 2) after the modeling, the administration the drugs (concentration: 0.1 mg/ml) started, 30 μl/eye/time, 3 times/day; On the tenth day (Day 11) after the administration, to observe the length and the numbers of corneal neovascularization (NV) distribution by clock directions on the rabbit eyes are and to calculate the corneal neovascularization area. The numbers are corrected based on acquired images using Photoshop CS, and the corneal neovascularization area is processed by Image Pro Plus; An area formula: S=C/12×3.1416×[R2−(R−L)2], the C represents the numbers by clock direction points when a corneal edge grows with the NV to non-NV in the picture, the R represents the length from an edge where a cornea contacts with a sclera to the center of the cornea in the picture, and the L represents the length from the root of new neovascularization of the edge where the cornea contacts with the sclera to the tail end of the NV in the cornea in the picture, and the longest neovascularization is acquired in each clock direction.

Test results:

| | | Left eye | | Right eye | |
|---|---|---|---|---|---|
| | | New | | New | |
| | | vascular | New | vascular | New |
| | | numbers by | vascular | numbers by | vascular |
| Animal | | clock | area | clock | area |
| No. | Group | direction | (S-mm²) | direction | (S-mm²) |
| 1 | Model | 9 | 81.57 | 8 | 66.35 |
| 2 | group | 4 | 30.65 | 9 | 71.24 |
| 3 | | 12 | 138.52 | 12 | 113.39 |
| 4 | Example 1 | 0 | 0.00 | 0 | 0.00 |
| 5 | group | 4 | 11.18 | 0 | 0.00 |
| 6 | | 0 | 0.00 | 3 | 14.98 |
| 7 | Example 4 | 6 | 34.80 | 7 | 25.12 |
| 8 | group | 0 | 0.00 | 0 | 0.00 |
| 9 | | 0 | 0.00 | 4 | 15.34 |
| 10 | Normal | 0 | 0.00 | 0 | 0.00 |
| | control | | | | |
| | group | | | | |

The experiment results show that administrated the drugs prepared in the Example 1 and 4 have very lower number and area of neovascularization than those of the model group, indicating that these two test drugs have a significant inhibition effect on the neovascularization.

### Example 11

### Animal Vitreous Body Absorption Experiment

The nanocrystalline eye drops prepared in Example 1-4 and the eye drops of the comparative example 1-4 are used to carry out animal vitreous body absorption experiments.

Sixty-six healthy male adult SD rats are selected, two for each group, a total of 33 groups. One group (4 eyes) is a blank control group, 40 μl of normal saline is dropped, and samples are taken 10 minutes after a sample liquid is added. The remaining 32 groups are testing groups, every 4 groups as one series, there is total of 8 series; to each series animals the nanocrystalline eye drops which are prepared in Example 1 to 4 and the comparative examples 1 to 4, are instilled 20 μl for each eye, respectively. The sampling time for each group in each series is set at different time point as 30 minutes, 60 minutes, 120 minutes and 240 minutes after instillation.

The specific sampling is to collect vitreous bodies of both eyes quickly after an animal is sacrificed by breaking a neck and store the vitreous bodies at −80° C. Thereafter, the vitreous body sample is homogenized, dilution, according to a standard sample preprocessing process with methanol or acetonitrile to obtain a liquid sample for liquid chromatography mass spectrometry analysis (LC/MS/MS) to determine the target compound concentration. LC/MS conditions: referring to SHIMADUZ No. C126. Sample analysis and process: an LC/MS/MS method is used to determine the drug concentration in the vitreous bodies, specific detection conditions are shown in table 4, and specific detection results are shown in table 5. However, no drug is detected in the vitreous body samples of comparative examples 1 to 4.

TABLE 4
Analytic Conditions
Chromatographic Conditions

| | SHIMADZU LC-20AD high performance |
|---|---|
| Apparatus | liquid chromatograph system |
| Chromatographic | INERTSIL ODS-3.5 μm 4.6 × 50 mm |
| column | |
| Mobile phase | Methanol: 5 mM ammonium acetate (containing |
| | 0.1% of formic acid) water (9:1, Wv) |
| Flow rate | 0.5 ml/min |
| Column temperature | 30° C. |
| Internal standard | XPS2497 (25 ng/ml) |
| Injection volume | 5 μl |
| Retention time | 1.47 min |

TABLE 5
Analytic Results

| | Single | Detection concentration | | | |
|---|---|---|---|---|---|
| | administration | (ng/mL) | | | |
| Sample No. | Time (h) | 0.5 | 1.0 | 2.0 | 4.0 |
| Example 1 | 20 μL/eye | 11.8 | 440 | 38.5 | 0 |
| Example 2 | | 26.1 | 3.5 | 5.2 | 0 |
| Example 3 | | 92.8 | 81.7 | 7.0 | 0 |
| Example 4 | | 0 | 771 | 278 | 43.3 |
| Comparative | | 0 | 0 | 0 | 0 |
| Example 1 | | | | | |
| Comparative | | 0 | 0 | 0 | 0 |
| example 2 | | | | | |
| Comparative | | 0 | 0 | 0 | 0 |
| example 3 | | | | | |
| Comparative | | 0 | 0 | 0 | 0 |
| Example 4 | | | | | |
| Control group | | 0 | 0 | 0 | 0 |

It can be seen from the table 5 that the nanocrystalline eye drops prepared in the examples of the present invention have good absorption, and the drugs can quickly pass through a blood-ocular barrier to enter the vitreous bodies, however, the eye drops prepared after changing the formulation or operation of the examples of the present invention cannot pass through the blood-ocular barrier to enter the vitreous bodies.

### Example 12

### Animal Pharmacodynamic Experiment

The pharmacodynamic study of a laser-induced mouse choroidal neovascularization (CNV) model

### 1) Sample Preparation

High-dose group: a sample with the drug concentration of 1 mg/ml prepared based on the conditions in the Example 13 (table 8 experiment conditions and result—No. 1);
Medium-dose group: 4 times dilution of the high-dose group;
Low-dose group: 4 times dilution of the medium-dose group.

### 2) Experiment Animal Preparation

Forty C57Bl/6c mice, 6-8 weeks old, 18-25 g, half male and half female, without abnormality in both eyes are chosen for laser-induced modeling.

Among them, the laser modeling refers to laser induction on fundi in both eyes of the mice to construct a CNV model, and the number of laser burns per eye is 3; laser parameters are wavelength 532 nm, power 120 mW, a light spot diameter 100 μm, and exposure time 100 ms.

The mice successfully modeled by laser photocoagulation are randomly divided into the following 4 groups:

TABLE 6
Experiment Conditions

| | | Administration | | |
|---|---|---|---|---|
| Group | | concentration | The number of animals | |
| No. | Group | (mg/ml) | Female | Male |
| 1 | Vehicle control group | 0 | 4 | 4 |
| 2 | Low-dose group | 0.0625 | 4 | 4 |
| 3 | Medium-dose group | 0.25 | 4 | 4 |
| 4 | High-dose group | 1.0 | 4 | 4 |

### 3) Dosing Frequency and Cycle

Eye drop instillation begins on the seventh day after modeling, 4 times/day, 5 μL/eye/time, for 14 consecutive days. Normal saline was administrated to the vehicle control group in the same manner.

Fundus photography (FP) is used to observe the retinal morphology of the fundi, and fundus angiography (FFA) is used to observe the leakage of the choroidal neovascularization.

### 4) Results

TABLE 7
Experiment Results
Improvement of light spot leakage of the
mouse CNV model by testing samples

| | Time | |
|---|---|---|
| | The average score of light spot leakage | |
| | | Two weeks after the |
| Subject | Before the administration | administration |
| Vehicle control group | 2.67 | 2.52 |
| Low-dose group | 2.63 | 2.06 |
| Medium-dose group | 2.61 | 2.13 |
| High-dose group | 2.61 | 1.83 |

Note:
the average score of light spot leakage = [(0-level light spot number × 0) + (1-level light spot number × 1) + (2-level light spot number × 2) + (3-level light spot number × 3)] ÷ 4 total light spots (that is, the number of effective light spots).

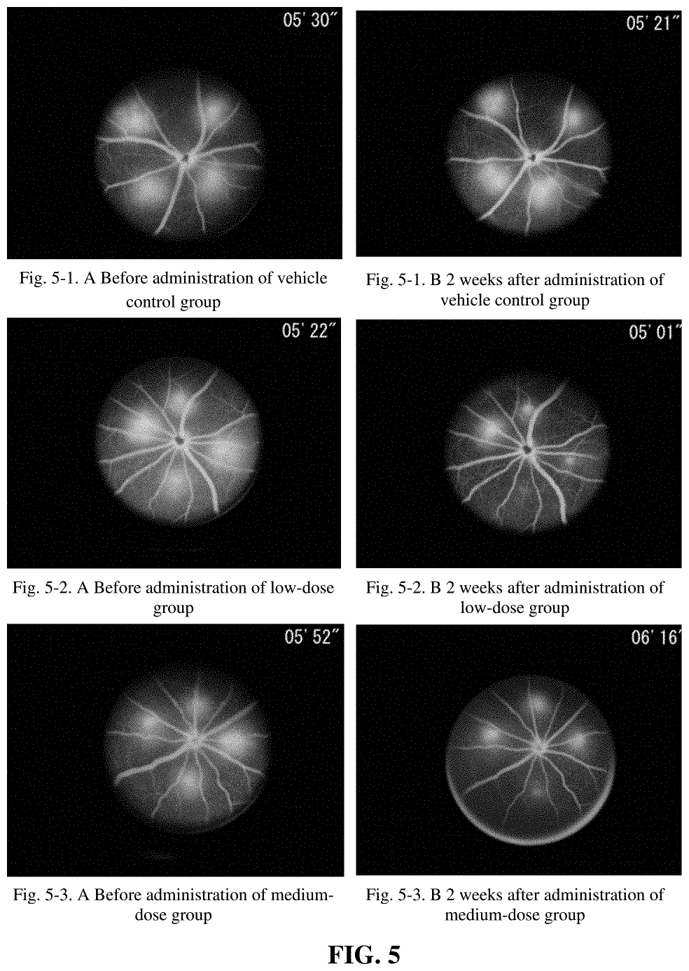

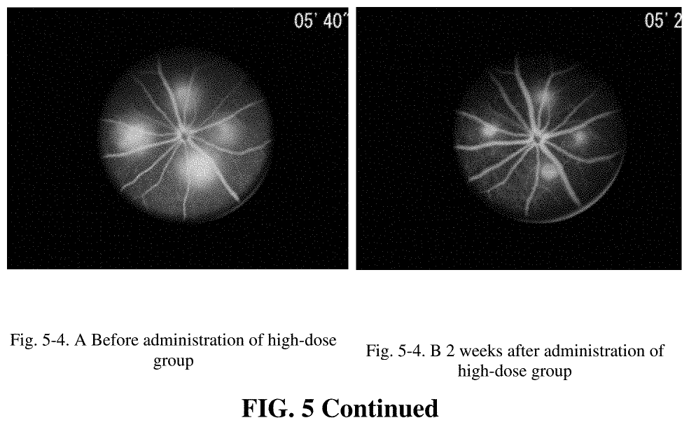

The fluorescein pictures of the animal eyes before and after the administration the nanocrystalline eye drops are shown in FIG. 5.

The experiment results show that, compared with the vehicle control group, the three testing groups of the present invention can reduce the eye spot light leakage of experimental animals, indicating that the nanocrystalline eye drops of the present invention can effectively reach the bottom of the eye and play a therapeutic role. It shows that the nanocrystalline eye drop according to the present invention can effectively reach the fundus and play a therapeutic role.

### Example 13

### Relationship Between the Drug Formulation and Animal Vitreous Body Absorption

In this example, different nanocrystalline eye drops are prepared and used for animal study to investigate the relationship between different nanocrystalline eye drops and the absorption of animal vitreous body.

The ball-milling method according to this example comprises the following steps:

1) respectively weighing, placing, and stirring double-soluble macromolecules and single-soluble macromolecule in a container containing 50 mL of purified water; heating in hot water bath (50-70° C.), continuing stirring till full dissolution;
2) weighing and placing a drug into the solution prepared in Step 1, starting a shearing machine to shear for 3-5 min at about 10000 rpm and get a preliminary suspension;
3) transferring the preliminary suspension prepared in Step 2 into a ball mill, where the ball-milling container is 100 ml sealing cup, and the grinding ball is ZrOZ beads with 0.3-0.4 mm (or 0.1-0.2 mm) in diameter; grinding for 2 h at 0° C.-10° C. and 350 rpm; filtering the obtained material by Buchner funnel and filter membrane under diminished pressure, and collecting the filtrate to obtain the nanocrystalline eye drop; recovering grinding ball.

The high-pressure homogenization method according to this comprises the following steps:

A. respectively weighing and placing double-soluble macromolecule and single-soluble macromolecule in a container containing 50 mL of purified water; stirring and heating (50-70° C. water bath) till full dissolution;
B. weighing and placing a drug into the solution prepared in Step A, starting a shearing machine to shear for 3-5 min at 10000-15000 rpm to prepare a preliminary suspension;
C. transferring the preliminary suspension prepared in Step B into a high-pressure homogenization machine, and controlling temperature to 5-10° C.; setting the pressure to not more than 1500 bar, and recycling for 15-20 times; finally recycling once at homogenization pressure of about 100-200 bar, discharging the solution, and performing membrane filtration to obtain homogeneous liquid.

The test method for drug content in rat vitreous body according to this example comprises the following steps:

selecting and grouping healthy adult SD rats with 4-6 eyes for each group; adding the prepared eye drop at 20 μl per eye dropwise, respectively; sacrificed the animals at set time point (1 h) to collect the vitreous body of both eyes and store the vitreous body at −80° C.; after homogenating, fully mixing the vitreous body with methyl alcohol or acetonitrile, filtration, obtaining filtrate as an analytical sample; determining the concentration of the target compound by a liquid chromatography-mass spectrometry (LC/MS), and calculating the API content in the test sample according to the API standard curve obtained under same analysis condition, as shown in Table 8.

TABLE 8
Experiment conditions and results

| | | | | Rat vitreous |
|---|---|---|---|---|
| | | | | concentration |
| S/N | Test Conditions | Test method | Properties | (ng/ml) |
| 1 | Targeting drug, axitinib 50 mg; | High pressure | Suspension | 92.8 |
| | Double-soluble macromolecule, Tween 80 | homogenization | | |
| | 0.5 g; Single-soluble macromolecule, HPC | method | | |
| | EF 0.5 g | | | |
| 2 | Targeting drug, axitinib 50 mg; | High pressure | Suspension | 20.3 |
| | Double-soluble macromolecule, Tween 80 | homogenization | | |
| | 0.5 g; Single-soluble macromolecule, HPC | method | | |
| | HF 0.5 g | | | |
| 3 | Targeting drug, axitinib 50 mg; | Ball-milling | Suspension | 67.8 |
| | Double-soluble macromolecule, poloxamer | method | | |
| | 0.5 g; Single-soluble macromolecule, HPC | | | |
| | HF 0.3 g | | | |
| 4 | Targeting drug, axitinib 50 mg; | Ball-milling | Suspension | 22.7 |
| | Double-soluble macromolecule, Tween 80 | method | | |
| | 0.5 g; Single-soluble macromolecule, | | | |
| | HPMC E5 0.3 g | | | |
| 5 | Targeting drug, axitinib 50 mg; | Ball-milling | Suspension | 14.4 |
| | Double-soluble macromolecule, Tween 80 | method | | |
| | 0.3 g; Single-soluble macromolecule, | | | |
| | HPMC E5 0.3 g | | | |
| 6 | Targeting drug, axitinib 5 mg; | High pressure | Suspension | 3.6 |
| | Double-soluble macromolecule, Tween 80 | homogenization | | |
| | 0.5 g; Single-soluble macromolecule, | method | | |
| | HPMC E5 0.3 g | | | |
| 7 | Targeting drug, axitinib 5 mg; | High pressure | Suspension | 42.3 |
| | Double-soluble macromolecule, Tween 80 | homogenization | | |
| | 0.5 g; Single-soluble macromolecules, HPC | method | | |
| | EF 0.3 g. | | | |
| 8 | Targeting drug, axitinib 12.5 mg; | Ball-milling | Suspension | 80.8 |
| | Double-soluble macromolecule, Tween 80 | method | | |
| | 0.3 g; Single-soluble macromolecules, HPC | | | |
| | EF 0.3 g. | | | |
| 9 | Targeting drug, axitinib 50 mg; | Ball-milling | Suspension | 74.0 |
| | Double-soluble macromolecule, Tween 80 | method | | |
| | 0.5 g; Single-soluble macromolecule, HPC | | | |
| | EF 0.5 g | | | |
| 10 | Targeting drug, axitinib 12.5 mg; | Ball-milling | Suspension | 70.9 |
| | Double-soluble macromolecule, Tween 80 | method | | |
| | 31 mg; Single-soluble macromolecule: HPC | | | |
| | EF 31 mg. | | | |
| 11 | Targeting drug, axitinib 0.05 g; | High pressure | Suspension | 32.5 |
| | Double-soluble macromolecule, poloxamer | homogenization | | |
| | 0.5 g; Single-soluble macromolecule, | method | | |
| | HPMC E5 0.3 g | | | |
| 12 | Targeting drug, Axitinib 5 mg; | High pressure | Solution | 1.7 |
| | Double-soluble macromolecule, Tween 80 | homogenization | | |
| | 2 mg; Single-soluble macromolecules, | method | | |
| | HPMC E5 2 mg. | | | |
| 13 | Targeting drug, axitinib 10 mg; | High pressure | Solution | 6.3 |
| | Double-soluble macromolecule, Tween 80 | homogenization | | |
| | 2 mg; Single-soluble macromolecules, | method | | |
| | HPMC E5 2 mg. | | | |
| 14 | Targeting drug, axitinib 50 mg; | High pressure | Suspension | 4.3 |
| | Double-soluble macromolecule, Tween 80 | homogenization | | |
| | 25 mg; Single-soluble macromolecule: | method | | |
| | HPMC E5 25 mg, CMC-Na 25 mg. | | | |
| 15 | Targeting drug, axitinib 5 mg; | High pressure | Suspension | 38.5 |
| | Double-soluble macromolecule, povidone | homogenization | | |
| | K30 150 mg; Single-soluble | method | | |
| | macromolecule, HPC HF 20 mg. | | | |
| 16 | Targeting drug, axitinib 5 mg; | High pressure | Suspension | 46.3 |
| | Double-soluble macromolecule, povidone | homogenization | | |
| | K30 100 mg; Single-soluble | method | | |
| | macromolecule, HPC EF 67 mg. | | | |
| 17 | Targeting drug, sunitinib 10 mg; | High pressure | Solution | 120.5 |
| | Double-soluble macromolecule, povidone | homogenization | | |
| | K30 100 mg; Single-soluble | method | | |
| | macromolecule, HPC HF 20 mg. | | | |
| 18 | Targeting drug, sunitinib 10 mg; | High pressure | Solution | 219.4 |
| | Double-soluble macromolecule, povidone | homogenization | | |
| | K30 100 mg; Single-soluble | method | | |
| | macromolecule, HPC EF 67 mg. | | | |

Note:
Water (50 ml) is used as the medium of the experiments in the table.

The result finally obtained by a great number of the experiment researches shows:

1) the double-soluble macromolecule and the single-soluble macromolecule have different chemical groups, polymerization patterns and polymerization degrees, which causes that they are very different in physicochemical property, lipid-water partition and stabilization for and biocompatibility with the targeting drug, etc. For example, compared with the eye drop prepared by using the hydroxypropyl methyl cellulose (HPMC E5), the eye drop prepared by using the hydroxypropyl cellulose (HPC μF or HF) as the single-soluble macromolecule and the Tween as the double-soluble macromolecule in the case of other conditions unchanged, is obviously better absorbed by the vitreous body of animals. The eye drop prepared by using the poloxamer+HPC HF is 1 time higher than that prepared by using poloxamer+HPMC E5 with respect to the absorbent concentration inside the vitreous body of animals;
2) the mass ratio between the double-soluble macromolecule and the targeting drug affects the absorption of the nanocrystalline eye drop inside the vitreous body of animals;
3) the final concentration of the double-soluble macromolecule or/and the single-soluble macromolecule in the eye drop will affect the absorption of the targeting drug; when the concentration of the double-soluble macromolecule is lower than 0.6 mg/ml, it is obvious to affect the drug absorption inside the vitreous body of animals;
4) such conditions as type, mass ratio and preparation technology of the targeting drug, the double-soluble macromolecule or/and the single-soluble macromolecule for preparing the nanocrystalline eye drop are different, which will result in different absorption of the prepared eye drop inside the vitreous body.

In conclusion, in the present invention, it is available to wrap the fat-soluble drug through interaction between the double-soluble macromolecule and the single-soluble macromolecule and further to form the nanocrystalline eye drop. Due to the hydrophily of the double-soluble macromolecule, the nanocrystalline eye drop is affiliative to the aqueous phase on the ocular surface. As the nanocrystalline eye drop is affiliative to lipid phase after contact with the ocular surface, it is helpful for the nanocrystalline eye drop to permeate the focus on fundus vitreous body. The nanocrystalline particle of drug is smaller, which is also good for penetratively enter the posterior segment.

Selecting small molecular tinib kinase inhibitors, the fat-soluble drug is as the preferred way in the present invention, since the small molecular tinib kinase inhibitor is easier to permeate into tissue than the macromolecular biological medicines. The pharmacodynamic study shows that the nanocrystalline eye drop of the present invention can ensure the effect of treating the neovascularization diseases of eye by using the targeting drug acting on VEGFR and/or PDGFR.

Through strict control for the types, mass ratio of the double-soluble macromolecule, the single-soluble macromolecule and the fat-soluble drug, and preparation technology, the present invention has the advantage that the prepared eye drop has stable property, is uneasy to accumulate or settle, and can fast go through the blood-ocular barrier to enter the fundus.

The above examples are part of examples of the present invention, but not all examples. The protection scope of the present invention is not limited by the detailed description of the examples according to the present invention, and these examples are only selected to describe the present invention. Based on the examples of the present invention, all other examples obtained by those skilled in the art without creative work shall fall within the protection scope of the present invention.

1. A nanocrystalline eye drop, characterized by comprising a double-soluble macromolecule, a single-soluble macromolecule and a fat-soluble drug;
the double-soluble macromolecule and the single-soluble macromolecule interact with each other to encapsulate the fat-soluble drug to form and stabilize a nanocrystalline.

2. The nanocrystalline eye drop according to claim 1, characterized in that the nanocrystalline eye drop is a solution or a suspension.

3. The nanocrystalline eye drop according to claim 1, characterized in that the fat-soluble drug comprises a targeting drug acting on vascular endothelial growth factor receptors and/or platelet-derived growth factor receptors.

4. The nanocrystalline eye drop according to claim 3, characterized in that the targeting drug comprises tyrosine kinase inhibitors,
preferably, the tyrosine kinase inhibitors are selected from any one or more of the tinibs and their medical acceptable salts;
more preferably, any one or more of axitinib, semaxanib, sorafenib, regorafenib, pazopanib, vandetanib, imatinib, nintedanib and sunitinib.

5. The nanocrystalline eye drop according to claim 1, characterized in that the double-soluble macromolecule is a macromolecular stabilizer containing both of hydrophilic group and lipophilic group, preferably a surface-active agent, more preferably any one or two of poloxamer, tweens, sodium dodecyl compounds, polyvinylpyrrolidone and polyethylene glycol compounds;
preferably, the sodium dodecyl compound is sodium dodecyl sulfonate and/or sodium dodecyl sulfate;
the polyethylene glycol compound is any one or more of PEG4000, PEG5000 or PEG6000.

6. The nanocrystalline eye drop according to claim 1, characterized in that the single-soluble macromolecule is a macromolecular suspending agent containing either hydrophilic group or lipophilic group, preferably any one or at least two of starch compounds, cellulose compounds or polycarboxylate compounds;
more preferably, the cellulose compound is any one or at least two of chitosan, hyaluronic acid, methyl cellulose, hydroxymethyl cellulose, hydroxypropyl cellulose, hydroxypropyl methyl cellulose and sodium carboxymethyl cellulose;
the starch compound comprises any one or at least two of sodium carboxymethyl starch, amylose and dextrin;
the polycarboxylate compound is any one or at least two of PLA, PGA and PLGA.

7. The nanocrystalline eye drop according to claim 1, characterized in that the particle size of the nanocrystalline in the nanocrystalline eye drop is 200-1000 nm, preferably 300-800 nm;
preferably, the mass ratio between the double-soluble macromolecule and the fat-soluble drug is 2-12:1 and preferably is 5-10:1;
preferably, the mass ratio between the double-soluble macromolecule and the single-soluble macromolecule is 1-5:1 and preferably is 1-2:1.

8. The nanocrystalline eye drop according to claim 1, characterized in that the content of the fat-soluble drug in the nanocrystalline eye drop is 0.06-100 mg/mL.

9. A method for preparing the nanocrystalline eye drop according to claim 1, characterized by comprising the following steps: mixing the double-soluble macromolecule, the single-soluble macromolecule and the fat-soluble drug, and then reducing the particle size of the drug to form a nanocrystalline encapsulated stably.

10. The preparation method according to claim 9, characterized in that the nanocrystalline eye drop is prepared by mixing the double-soluble macromolecule and the single-soluble macromolecule to form a mixed solution;
then, mixing the mixed solution and the fat-soluble drug to form an initial suspension;
and then, grinding or homogenizing the initial suspension to form the nanocrystalline eye drop that stably encapsulates the fat-soluble drug.

11. The preparation method according to claim 9, characterized by comprising: mixing the double-soluble macromolecule, the single-soluble macromolecule and water to form the mixed solution and then mixing the mixed solution and the fat-soluble drug to form the initial suspension.

12. The preparation method according to claim 11, characterized in that a dosage of the double-soluble macromolecule is 4-1000 mg in every 100 mL of water in the mixed solution;
and/or a dosage of the single-soluble macromolecule is 4-1000 mg in every 100 mL of water;
preferably, the dosage of the double-soluble macromolecule is 10-300 mg in every 100 mL of water.

13. An application of the nanocrystalline eye drop according to claim 1 in preparation of a drug used for treating fundus oculi diseases or/and ocular surface diseases.

14. The application according to claim 13, the fundus oculi diseases comprising diseases related to fundus neovascularization and the ocular surface diseases comprising diseases related to ocular surface neovascularization;
preferably, the diseases related to fundus neovascularization comprise any one or more of age-related macular degeneration, retinal vein occlusion macular edema, central retinal vein occlusion, diabetic retinopathy, diabetic macular edema, or impaired vision, neovascular glaucoma and eye tumors caused by choroidal neovascularization secondary to pathological myopia;
preferably, the diseases related to ocular surface neovascularization comprise any one or more of viral keratitis, corneal neovascularization caused by physical and/or chemical trauma, corneal transplantation, corneal neovascularization, ocular surface neovascularization and pterygium, corneal neovascularization complicated by pterygium, corneal neovascularization due to corneal transplantation rejection, and deficiency of corneal stem cells.
